(19) United States
(12) Patent Application Publication
(10) Pub. No.: US 2025/0372234 A1
(43) Pub. Date: Dec. 4, 2025

(54) READING ERROR REDUCTION BY MACHINE LEARNING ASSISTED ALTERNATE FINDING SUGGESTION

(71) Applicant: KONINKLIJKE PHILIPS N.V., EINDHOVEN (NL)

(72) Inventors: JOËL VALENTIN STADELMANN, MOSCOW (RU); HEINRICH SCHULZ, HAMBURG (DE)

(21) Appl. No.: 18/874,827

(22) PCT Filed: Jun. 5, 2023
(86) PCT No.: PCT/EP2023/064919

§ 371 Date: Dec. 13, 2024

(30) Foreign Application Priority Data
Jun. 14, 2022 (RU) 2022115966

(51) Int. Cl. G16H 30/20; G16H 10/60

(52) U.S. Cl. CPC G16H 30/20; G16H 10/60

(57) ABSTRACT

A pre-processor (PP) component and related method for a machine learning system (MLS) for processing medical data. The preprocessor comprises an input interface (IN) for receiving a human generated initial finding for a patient and a medical image to which the said finding pertains. An encoder (ENC) DPS of preprocessor encodes the finding and the medical image into encoded data, including encoded image data and encoded finding data. A combiner (COM) component of preprocessor combines the encoded finding and the encoded image data into combined encoded data. An output interface (OUT) provides the combined encoded data to the machine learning system. More robust machine learning performance may be achieved with the proposed pre-processor (PP).

### FIELD OF THE INVENTION

The invention relates to a pre-processor component for a machine learning model for processing medical data, to a related method, to a machine learning arrangement, comprising the pre-processor component and the machine learning model, to a training system for training machine learning model, to a method of training machine learning model, to a computer program element and to a computer readable medium.

### BACKGROUND OF THE INVENTION

The interpretation (referred to herein as “reading” or “review”) of radiological studies is a difficult task.

It is estimated, that at least 5% of the patients experience some form of diagnostic error, that contribute to up to 17% of hospital adverse errors.

Around 75% of those errors are centered around radiology practice. Most of the research dedicated to the prevention of radiological errors concentrates around the avoidance of false negatives, with a special attention drawn to the radiologist's fatigue and its influence on image perception and interpretation, and his or her working ergonomics.

However, to date, little is done to verify the correctness of a radiologist's interpretation, as it is assumed that the radiologist is in possession of sufficient information to provide a correct interpretation, if it was not due to fatigue.

There are, however, literature reports, that demonstrate that a radiologist's interpretation may not always be correct, even if there is no fatigue. Certain radiology findings can easily be confused for one another. Examples reported in literature include lymphoma misinterpreted as hematoma, or confusion between the different causes of lung consolidation such as water-transudate, pus-exudate, blood-hemorrhage, or cells-tumor/chronic inflammation.

Causes of misinterpretation range from an inadequate radiology experience, confusing patient history or incorrect imaging. The consequences or image reading errors include the need of reimaging, unnecessary surgical operations, or even patient death.

### SUMMARY OF THE INVENTION

There may therefore be a need for facilitating reduction of error rate in image readings.

An object of the present invention is achieved by the subject matter of the independent claims where further embodiments are incorporated in the dependent claims.

It should be noted that the following described aspect of the invention equally applies to the related method, to the machine learning arrangement, comprising the pre-processor component and the machine learning model, to the training system for training machine learning model, to the method of training the machine learning model, to the computer program element and to the computer readable medium.

According to a first aspect of the invention there is provided a pre-processor component for a machine learning system for processing medical data, comprising:
at least one input interface for receiving a human generated initial finding for a patient and a medical image to which the said finding pertains,
an encoder for encoding the finding and the medical image into encoded data, including encoded image data and encoded finding data;
combiner component for combing the encoded finding and the encoded image data into combined encoded data, and
an output interface for providing the combined encoded data to the machine learning system.

In embodiments the input interface is to receive contextual data, providing context information in relation to the report and/or the image, the encoder configured encode at least a part of the contextual data into the encoded data, and the combiner to combine the encoded contextual data with the image and the encoded report to obtain the combined data.

In embodiments, the contextual data includes any one or more of: i) the patient history, ii) imaging request for the image, iii) statistical data in relation to misdiagnosis.

In embodiments, the combiner and/or the encoder is implemented as a machine learning model.

In embodiments, the machine model for the encoder includes a processing channel configured for recurrent processing.

In embodiments, the processing channel is configured to process at least the encoded patient history.

In embodiments, an expected dimensional size of encoded patient history is variable.

In embodiments, the encoded data includes at least one of: at least one of a matrix or at least one vector. This allows efficient computational implementation.

In embodiments, the at least one vector includes a one-hot vector, but other coding techniques may be used instead.

In another aspect there is provided a machine learning arrangement comprising the pre-processor component according to any one of the above mentioned embodiment, and the machine learning system.

In embodiments, the machine learning system includes a machine learning model configured to transform the combined encoded output into output data that is indicative of at least one second finding, the second finding being either an alternative to the initial finding or being equal to the initial finding.

In embodiments, the output includes: natural textual string or a medical finding code.

In embodiments the arrangement includes a localizer configured to map the output data to an image location in the image.

In another aspect there is provided a training system configured to train based on training data the machine learning model of any one or both of the mentioned embodiments.

In another aspect there is provided a method of pre-processing medical data for machine learning, comprising:
receiving a human generated initial finding for a patient and a medical image to which the said finding pertains,
encoding the finding and the medical image into encoded data, including encoded image data and encoded finding data;
combing the encoded finding and the encoded image data into combined encoded data, and
providing the combined encoded data to the machine learning system.

In another aspect there is provided a method of processing the provided combined encoded data by a further machine learning model. Specifically, this method may include transforming the combined encoded output into output data that is indicative of at least one second finding, the second finding being either an alternative to the initial finding or being equal to the initial finding.

In another aspect there is provided a method of training, based on training data, the machine learning model of any one of all of the above mentioned embodiments or aspects.

In another aspect there is provided, a computer program element, which, when being executed by at least one processing unit, is adapted to cause the at least one processing unit to perform the pre-processing method or the training method.

In another aspect there is provided at least one computer readable medium having stored thereon the program element, or having stored thereon the machine learning model.

Medical findings are decisions in respect of medical conditions that are taken based on partially available information. For example, such as decision may be formulated as “Does this 47 year-old patient have a heart attack?”. The context data allows adding potentially relevant information (such as “patient is male” and/or “has a history of heavy smoking”, etc). By using such contextual data, the decision process becomes more robust, but may come at a cost of speed or computational resources. However, there exist (irrelevant) information that does not improve the system's robustness, while still costing processing time. The pre-processor as proposed herein preprocesses information from different sources in order to balance this information according to its relevance, so as to improve its pertinence to the desired output (the finding). The adverse effect on computing time is mitigated by preferably parallelizable algorithms, that can be run on specialized hardware such as GPU or other.

The encoded combined findings produced by pre-processor are preferably elements of a vector space. The encoded combined findings include preferably encoded contributions from plural (such as all) data types originally received, such as the input image, the initial finding and, optionally, one or more of the contextual data. This represents a balancing the input data which can be more robustly processed by the transformer so as selectively rebalance the relevance of the varies data type for the finding to be computed.

The pre-processor may be used in a proposed machine learning (“ML”) module working alongside the radiologist. preferably in real-time, to suggest for example alternative interpretation(s)/findings as he or she is filling the report, thus reducing reading errors.

The ML based recommendation module as proposed herein in embodiments analyses the radiologist's interpretation of a study and suggests different possible readings which are brought to radiologist's attenuation. With this option for double-checking, reading error could be reduced. By helping to reduce reading errors, costs can be reduced. The current cost of misdiagnoses is a staggering 17 to 29 billion USD annually, costs which the health sector could spend elsewhere with much more benefit. There is the expectation that such reading error reduction translates into overall better patient care by reducing the number of re-imaging procedures, or of unnecessary interventions caused by misdiagnosis.

The proposed ML module is capable of processing radiologist reports in free-hand written form, or in any type of unstructured form. A structured report such as a table, a checkmarkable list etc., is not required herein.

The proposed system and method can be applied to all kinds of radiology modalities, for instance chest X-ray, CT, MRI, PET or ultrasound studies.

Whilst use of the pre-processor in such module is preferred herein, such use is not at the exclusion of other uses, including stand-alone uses, where the data of the pre-processor may be used on its own, such as in medical data analytics to explore interrelationships between data from different sources.

“user” relates to a person, such as medical personnel or other, operating the imaging apparatus or overseeing the imaging procedure, conducting the image review/reading sessions, such as a radiologist. In other words, the user is in general not the patient.

In general, the term “machine learning” includes a computerized arrangement (or module) that implements a machine learning (“ML”) algorithm. Some such ML algorithms operate to adjust a machine learning model that is configured to perform (“learn”) a task. Other ML operate direct on training data, not necessarily using such as model. This adjusting or updating of training data corpus is called “training”. In general task performance by the ML module may improve measurably, with training experience. Training experience may include suitable training data and exposure of the model to such training data. Task performance may improve the better the data represents the task to be learned. Training experience helps improve performance if the training data well represents a distribution of examples over which the final system performance is measured. The performance may be measured by objective tests based on output produced by the module in response to feeding the module with test data. The performance may be defined in terms of a certain error rate to be achieved for the given test data. See for example, T. M Mitchell, “Machine Learning”, page 2, section 1.1, page 6, section 1.2.1, McGraw-Hill, 1997.

### BRIEF DESCRIPTION OF THE DRAWINGS

Exemplary embodiments of the invention will now be described with reference to the following drawings, which, unless stated otherwise, are not to scale, wherein:

FIG. 1 is a block diagram of a medical arrangement for processing medical data including medical imagery;

FIG. 2 is a machine learning module that may be used in the arrangement of FIG. 1 and is capable of producing an alternative medical finding alternative to a finding in input data receivable for processing by the module;

FIG. 3 shows a block diagram of an architecture of the machine learning module in FIG. 2;

FIG. 4 shows a computer implemented training system for training a machine learning model that may be used in the module of FIG. 2 or 3;

FIG. 5 shows a flow chart of a computer implemented method of processing medical data, in particular for computing an alternative finding that is alternative to a finding included in initial input data; and

FIG. 6 shows a flow chart of a computer implemented method for training a machine learning model.

### DETAILED DESCRIPTION OF EMBODIMENTS

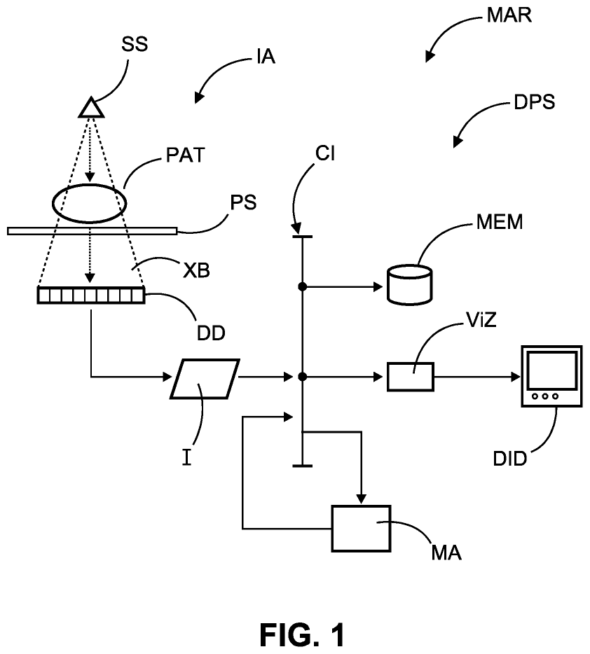

Reference is now made to FIG. 1 which shows a block diagram of a medical arrangement MAR for processing medical data, in particular measurements in respect of a patient. Broadly, the arrangement includes a medical measurement set-up, such as a medical imaging apparatus IA, that produces measurements with respect to the patient.

The measurement taken in respect of the patient may include medical imagery. Based on the medical imagery, a computer-implemented medical recommender module MA, preferably machine learning implemented, is operative to compute one or more alternative findings that are alternative to finding(s) provided by a human medical user. Operation of the medical recommender module MA is based on the user provided initial finding, and on the measurements, in particularly on imagery I, on which the user's initial finding was based.

Thus, the medical recommender MA is configured herein, preferably based on machine learning, to operate alongside a radiologist to suggest alternative findings that are alternative to the one(s) the human radiologist has arrived at when examining the same imagery in respect of the same patient. The user activity in examining the measurements, such as imagery, in order to arrive at his or her finding is called “reading”. Broadly then, the recommender module MA helps reducing a risk of errors in reading medical measurements, in particular in reading imagery. In the following we will refer exclusively to medical imagery as one example of such medical measurements, with the understanding that the principles described herein are of equal application to other types of medical measurements, such as laboratory data (eg, blood samples), ECGs, EEGs, or any other such medical data that describes patient's (medical) state.

Thus, the machine learning recommender MA operates to analyze the imagery and, in addition thereto, the initial findings of medical user (such as radiologist) to derive/infer possible alternative finding (s), if any. If there are no alternative findings, that is, if the findings derived by the recommender module MA are identical or sufficiently similar to the one(s) provided by the radiologist, this fact may be flagged up suitably by a confirmation signal or other. If the machine learning recommender MA generated finding(s) differs from the user's initial finding, so is an alternative finding, and this fact too may be indicated graphically, numerically or in any other form on the display device DID or by using any other suitable transducer. Alternatively, or in addition to visualization, the alternative findings computed by the recommender may be stored or otherwise processed. In the following we will refer to “finding” in the singular, with the understanding that there may be multiple findings involved, either as generated by recommender MA or by user. Thus, a reference herein to “finding” does not necessarily mean a single finding (although this is not excluded herein), but should be construed as a reference to “at least one finding”. In general, as understood herein, “finding” is a specification in medical terms, either in coded form or in natural language or graphically, etc, that describes one or more aspects of a patient's medical state. Thus, a finding may include an indication of disease, condition, ailment, etc, in respect of patient, or an absence thereof (“patient is healthy”).

Before explaining operation of the arrangement MAR in more detail, and in particular of the recommender module MA, some components of the imaging apparatus IA will be explained first. Generally, the imaging apparatus IA may include a signal source SS and a detector device DD. The signal source SS generates a signal, for example an interrogating signal, which interacts with the patient to produce a response signal which is then measured by the detector device DD and converted into measurement data such as the said medical imagery. One example of the imaging apparatus or imaging device is an X-ray based imaging apparatus such as a radiography apparatus, configured to produce protection imagery. Volumetric tomographic (cross sectional) imaging is not excluded herein, such as via a C-arm imager or a CT (computed tomography) scanner, or other.

During an imaging session, patient PAT may reside on a patient support PS (such as patient couch, bed, etc), but this is not necessary as the patient may also stand, squat or sit or assume any other body posture in an examination region during the imaging session. The examination regions is formed by the portion of space between the signal source SS and the detector device DD.

For example, in a CT setting, during imaging session, X-ray source SS rotates around the examination region with the patient in it to acquire projection imagery from different directions. The projection imagery is detected by the detector device DD, in this case an x-ray sensitive detector. The detector device DD may rotate opposite the examination region with the x-ray source SS, although such co-rotation is not necessarily required, such as in CT scanners of 4^{th} or higher generation. The signal source SS, such an x-ray source (X-ray tube), is activated so that an x-ray beam XB issues forth from a focal spot in the tube during rotation. The beam XB traverses the examination region and the patient tissue therein, and interacts with same to so cause modified radiation to be generated. The modified radiation is detected by the detector device DD as intensities. The detector DD device is coupled to acquisition circuitry such as DAQ to capture the projection imagery in digital for as digital imagery. The same principles apply in (planar) radiography, only that there is no rotation of source SS during imaging. In such a radiographic setting, it is this projection imagery that may then be examined by the radiologist. In the tomographic/rotational setting, the muti-directional projection imagery is processed first by a reconstruction algorithm that transforms projection imagery from projection domain into sectional imagery in image domain. Image domain is located in the examination region. Projection imagery or reconstructed imagery will not be distinguished herein anymore, but will simply be referred to collectively as (input) imagery I or input image(s) I. It is such input imagery I that may be processed by recommender MA.

The input imagery I may however not necessarily result from X-ray imaging. Other imaging modalities, such as emission imaging, as opposed to the previously mentioned transmission imaging modalities, are also envisaged herein such as SPECT or PET, etc. In addition, magnetic resonance imaging (MRI) is also envisaged herein in some embodiments.

In MRI embodiments, the signal source SS is formed by radio frequency coils which may also function as detector device(s) DD, configured to receive, in receive mode, radio frequency response signals emitted by the patient residing in a magnetic field. Such response signals are generated in response to previous RF signals transmitted by the coils in transmit mode. There may dedicated transmit and receive coils however in some embodiments instead of the same coils being used in the said different modes.

In emission imaging, the source SS is within the patient in the form of a previously administered radio tracer which emits radioactive radiation that interacts with patient tissue. This interaction results in gamma signals that are detected by detection device DD, in this case gamma cameras, arranged preferably in an annulus around the examination region where the patient resides during imaging.

Instead of, or in addition to the above mentioned modalities, ultrasound (US) is also envisaged, with signal source and detector device DD being suitable acoustical US transducers.

The imagery I generated by whichever modality IA may be passed through a communication interface CI to a (non-volatile) memory MEM, where it may be stored for later review or other processing. However, an online setting is not excluded herein, where the imagery is reviewed as it is produced by the imaging apparatus. Having said that, in many occasions, an offline setting may be sufficient or more apt, where the imagery is first stored in the said memory MEM, preferably in association with the respective patient's ID. The image memory may be non-volatile, such as a medical image data base of the likes of a PACS or similar. Once user wishes to review (or “read”), the stored imagery of a patient of interest is accessed by suitable database query system using the patient's ID for example or other indicia. The accessed imagery may be passed to a viewer software VIZ. The viewer software may be operative to produce visualization of the imagery as a graphics display, which is then displayed on a display device DID. The above-mentioned reviewing may be done on any suitable computing platform, mobile (laptop, desktop, tablet) or stationery (desktop, workstation etc) on which the visualizer VIZ and the DB query system may be run or from which it can be controlled whilst running remotely on a server for example.

The radiologist can review the imagery in one or more review sessions. The radiologist may enter his or her findings in medical terms into a structured or free text report file. The report file is a data structure that resides in a computer memory of the reviewing system (not shown). The report file includes data (such as text strings, codes, etc) that is indicative of the finding(s). The report file will be referred to herein simply as “the report”. The report may be stored preferably in association with the imagery on which the review was based and/or in association with the respective ID of patient of whom the imagery I was taken. This allows convenient retrieval of all relevant information at a later time, should the need so arise, such as during reviewing. The report may be stored in the same database as the imagery, or may be stored in a different data base such as patient record data base (a HIS or other).

Human reviewers are often under stress and must read large amounts of image material, such as in a busy clinic with an incessant in-flow of new patients, some of whom may be trauma-patients, requiring immediate attention. In such environments with high stress and high work-load, reading errors may occur where reviewer may, unfortunately, associate wrong findings with the imagery, with potentially disastrous consequences for the patient. Errors of the false-positive, or even worse, false-negative type may come up this way. Such occurrences of wrong readings may be due to user fatigue and/or may be brought about by poor quality imagery. For example, crucial image information may be occluded, for example by artifacts or by ill-placed tags, text information boxes, annotations or the like, or other such widgets later added into the imagery by superposition for example.

Examples of such wrong readings or misdiagnoses, include lymphoma misreported as hematoma, or confusion between the different causes of lung consolidation: water-transudate, pus-exudate, blood-hemorrhage, or cells-tumor/chronic inflammation. Causes of misinterpretation may be manifold. They may range from an inadequate radiology experience, to confusing patient history, and incorrect imaging, to name just a few examples.

In order to reduce the likelihood of such wrong readings, the recommender module MA processes the radiologist's report (that includes the initial finding) together with the input imagery I to compute an alternative finding. The initial finding and the imagery to which the finding pertains form core input data c.

The alternative finding computed by module MA may be brought to the attention of the radiologist as mentioned above. The alternative finding may be sounded out or may be displayed instead of or alongside the original/initial finding as previously assigned by the radiologist. The radiologist may then choose to accept the alternative finding, thus correcting the original finding, or the radiologist may choose to reject the alternative finding and maintain the original finding. Such acceptance and rejection operation may be done though a suitable user interface (UI) arrangement, such as via a keyboard, touch screen, pointer tool (stylus, computer mouse), etc. Instead of such an alternative finding, the original finding may be confirmed as mentioned earlier.

Preferably, the recommender MA operates whilst the radiologist is reviewing the visualized imagery. The visualized imagery is produced by visualizer VIZ on display device DID for example. The recommender module MA may thus interact with visualizer VIZ to provide the alternative or confirmatory finding in a suitable visualization, preferably concurrent with the image that is reviewed, and/or concurrent with the displayed report.

As will be explained in more detail below, in some embodiments the alternative finding may be localized by localizer LC in the imagery on which the review was based. The localizing may be implemented by displaying an overlay widget, such as a bounding box, or other graphical indicator widget, to indicate the portion in the input image I to which the alternative finding pertains. The alternative finding may be indicated as text, in code, in free text or otherwise, preferably displayed in relation to the bounding box, such as inside the bounding box or adjacent to it or in any other spatially associable manner.

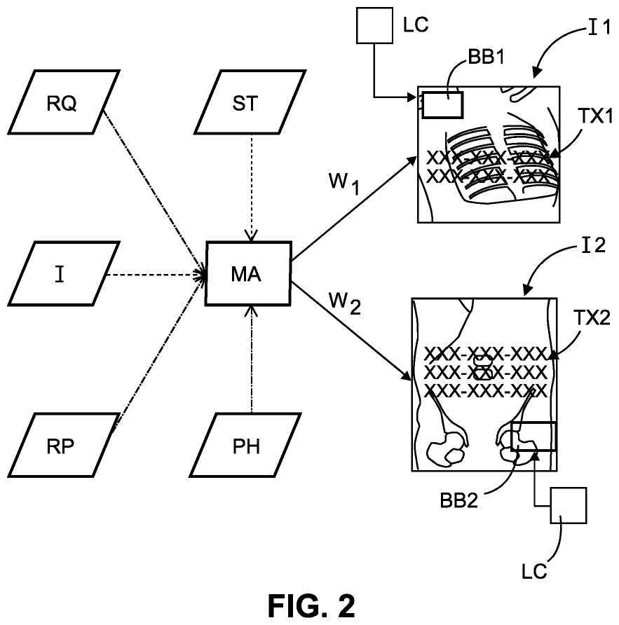

Operation of recommender module MA is now shown in block diagram of FIG. 2 in more detail.

As can be seen, the recommender MA processes input data. Input data includes the core input data comprising the image I which was reviewed by the human reviewer, and the original/initial finding such as included in the radiologists report RP. The report RP may include textual or other information that is indicative of the finding that the radiologist arrived at based on the said input image I. Input image I and the report RP pertain to the same patient. It is not necessary herein to include the report RP as such in the core input data although this is envisaged herein in some embodiments. Instead, it may be sufficient to extract the original finding from the report RP by an NLP (natural language processing) pipeline, string matching or other textual or string processing, and to include the so extracted data indicative to the finding in the input core data. The NLP pipeline itself may be implemented by a dedicated machine learning model, such as a BERT type NN model, or other fully connected architecture configured for natural language processing.

Optionally, contextual data is processed alongside the core input data c as contextual input data x. The contextual input data x provides context in relation to the patient and/or the finding. This can help make the machine-based recommender MA operate more robustly. The contextual data x can act as a regularizer during training. Training aspects are described later below in more detail.

The contextual data x may include a number of different data items, either singly or in any combination. For example, the contextual data x may include the request RQ for the radiological study which resulted in the input image being produced. This request includes information that has led the radiologist or other medical user to request the imaging session during which the input image was produced. The imaging RQ data may include text data in a structured or unstructured format which represents the reasons for requesting imaging for image I. Typically, the imaging request data RQ may include suspected diagnostic clues based on which the imaging session to produce the image I by modality IA was requested.

Alternatively or in addition, the contextual data x may include patient reference data PH that includes the patient's medical history, such as patient records, prior imagery, etc, or extracts therefrom. This medical history data PH can be pulled from databases where this information concerning the patient at hand may be stored.

In addition or instead still, the contextual data x may include statistical data ST or statistical analysis of previous mis-diagnoses. This data type relates to previous cases, not necessarily in respect of the instant patient but to a cohort of other patients, where mis-diagnosis/wrong readings occurred, that is, where the original finding produced by the radiologist was later found in fact to be wrong.

This type of data ST may typically include the correct finding as may have been found by later investigation which typically takes place when it emerges that in fact the original finding was wrong. Thus, review data of mis-diagnosed cases includes valuable information where user produced findings are correlatable with their respective correct finding. Thus, it can be derived for example from such data by statistical analysis, which type of (diagnostic) findings are more prone to a mis-diagnosis than others. The core input data c (including imagery I and initial finding such as in report RP), preferably enriched by contextual data x=(request RQ, statistics ST, patient history PH) may be processed by machine learning based module MA to produce for example one or more alternative findings W1, W2. Again, not necessarily all the described contextual data x may be used. Any sub-combination of the above is envisaged herein. However, it is in particular inclusion into context data x of the imaging request RQ and/or of the statistical data ST that have been found to yield good results. An example could be a trauma patient: findings may include “bone fractures”, “organ laceration”, and “internal bleeding”. The radiologist reports trauma, as should be. However, it's April 2020, the statistic trends may map out effects of, for example, COVID 19, and the exhausted personal sent the request as a chest X-ray for an infectious patient. In this case, the system should discard or down-weight the context, and not provide a “pneumonia” recommendation. After five days, when the patient starts developing a pneumonia on a control X-ray, the system should ignore or down-weight the context that the patient is a trauma patient and consider the finding of an infectious disease trauma patient.

The visualizer VIZ may indicate the finding W1,W2 in textual form TX1,TX2. Two such findings are shown but there may be more or less than two. The alternative findings may be superimposed on the input imagery in question, such as on imagery I1, I2 as exemplary shown to the right of FIG. 2. Bounding boxes BB1 and BB2 may be used to indicate the portion of the image which contributed more than other image portions in the course of computations performed by the machine learning module MA when producing the alternative finding W1,W2. Hot-map technology may be used to derive at the bounding boxes as will be detailed below. However, any other localization technique may be used, for example where the input data includes explicit co-ordinates indicating the portions of the image in support of the findings and such coordinates are learned alongside the imagery. Localization may be achieved by a localizer LC, operation of which may be explained in more detail below.

The textual representation TX1 and TX2 of the alternative findings W1 and W2 may include textual strings, such as in tree text form, natural language text, or may be in the form of medical terminology coding, such as WHO (World Health Organization)'s ICD (International Classification of Diseases) coding, such as version 11 or earlier versions, or future versions. However, any other suitable medical coding may be used instead. in general, coding as used herein is distinct from encryption, the latter being irreversible without knowledge of crypto-key(s), whilst the earlier is reversable with no such keys required, with lower computational burden.

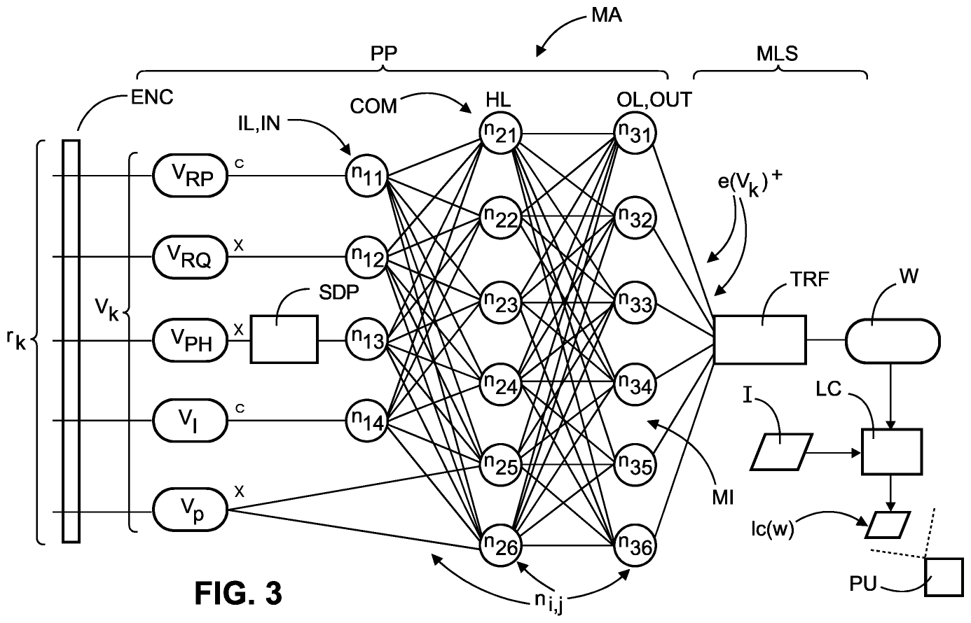

Reference is now made to the block diagram in FIG. 3 which shows yet more details of the machine learning based recommender module MA. Conceptionally, there is a latent relationship between the input data (that is, the core data c, potentially enriched with the contextual data x), and its associated alternative finding. This relationship can be thought to be implicitly expressed in existing training data including historical imagery and related historical patient records. Such training data could be found in medical databases, and may relate to prior examinations for a suitably representative cohort of patients.

The latent relationship may be difficult, even impossible, to model classically, analytically in an ad-hoc manner. To this end, machine learning models are used that do not require such explicit modelling. Standard models with parameters can be used that are adapted in a learning/training process, based on the training data. The parameters may be adjusted in iterations, until the adapted model is deemed (in pre-defined manner based on a cost function) a good enough approximation of the said latent relationship. Aspects of learning will be described in more detail below.

With continued reference to FIG. 3, and in more detail, this Figure represents a model architecture that may be used to build recommender module MA. The model is capable of computing, after suitable training on training data, the alternative findings (if any), given at least the core input data c as described above. Broadly, model for recommender MA may include a pre-processor PP and, downstream thereof, a post-processor, the latter referred herein as the recommender machine learning system MLS.

Both, the pre-processor PP and the post-processing recommender machine learning system MLS, may each be implemented by a dedicated respective machine learning model as will be described in more detail below.

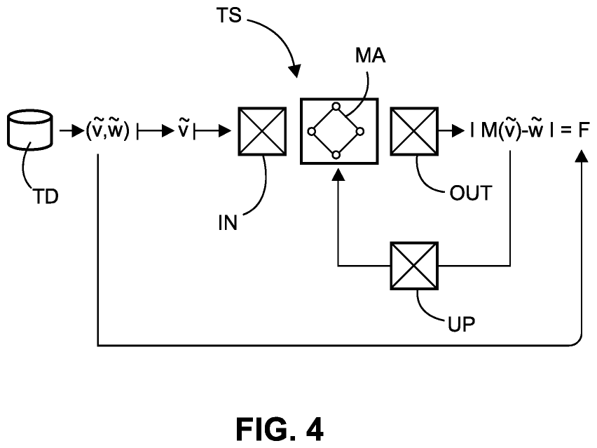

Broadly, post-processor PP processes input data vₖ (which include the core data c⊂vₖ), either in training, in testing or in deployment (that is, in real-world clinical post-training application), and computes intermediate output e(vₖ). Intermediate output is passed on to post-processor stage MLS which then transform the intermediate output into the desired finding, such as an alternative finding w, if any, or confirmatory finding. Pre-processor PP and/or post-processor MLS may be implemented as respective trained machine learning models. In the following it is assumed that the models have been trained. Aspects of training with be described in more detail at FIGS. 4,6.

In yet more detailed reference to pre-processor PP, raw input data rk is applied to an encoder ENC portion of pre-processor PP. The raw data is in digital form, and may be the result of conversion into such digital data by data capture, A/D conversion, and character recognition, etc. For example, a handwritten report may be captured as an image and then OCR processed. Alternatively, the report or other text data is generated by word-processing module, etc. The raw data is encoded by encoder ENC into input data vk for a second stage of pre-processor PP. This second stage is configured as a combiner COM. The encoded input data vₖ for combiner COM includes core data c and, optionally, context data x. The combiner COM combines the encoded input data vk into combined encoded data e(Vk)⁺.

Combiner COM of pre-processor PP may be configured as set of computational nodes nᵢⱼ of an artificial neural network (“NN”). Each node is associated with parameters (“weights”), previously adapted in training based on training data. The nodes nᵢⱼ may be arranged in a cascaded fashion in layers. In some embodiments, the combiner COM is arranged as a convolutional neural network (“CNN”), the computational nodes implementing convolutional operators.

The layers may include input layer IL, IN, one or more hidden layers HL, and an output layer OL, OT. Encoded input data is supplied vₖ to the input layer, processed there and propagated through the one or more layers hidden layer(s) HL, to the output layer OL. The output of the output layer OL may include feature maps that represent intermediate data which represents encoded data e(vₖ) of input vₖ. As an illustration only, an architecture with 3 layers is shown in FIG. 3, but there may be more than three. It has been observed that a rather shallow network with merely a single hidden layer as shown in FIG. 3 performs well. Keeping the network shallow with a single or a few (such as 2, 3 or 4) hidden layers allows for high responsiveness as fewer computations are required.

The input and output between hidden layers HL, and the output of input layer IL and of output layer OL may be referred to herein as feature maps. Feature maps can be represented as two or higher dimensional matrices (“tensors”) for computational and memory allocation efficiency.

Preferably, some or all of the layers IL, HL are convolutional layers, that is, include one or more convolutional filters which process an input feature map from an earlier layer into intermediate output, sometimes referred to as logits. An optional bias term may be applied by addition for example. An activation operator of given layer processes in a non-linear manner the logits into a next generation feature map which is then output and passed as input to the next layer, and so forth. The activation operator may be implemented as a rectified linear unit (“RELU”), or as a soft-max-function, a sigmoid-function, tanh-function or any other suitable non-linear function. Optionally, there may be other functional layers such as pooling layers or drop-out layers to foster more robust learning. The pooling layers reduce dimension of output whilst drop-out layer sever connections between node from different layers. The combined or “hybrid” encodings e(vk)⁺ produced by combiner COM are in embodiments such one or more feature maps. Preferably, the encodings e(vk)⁺ equals the number of encoded data streams (vk) as feed into input layer IL. However, there may be more or less of such combined encodings e(vk)+.

The computational function of nodes nᵢⱼ are in general (weighted) linear combinations of the logits from previous layers, and also include applying the non-linearity to the so combined logits. However, other functional combinations, not necessarily linear combinations, are also envisaged.

The post-processor MLS may include a transformer TRF stage. The transformer is operative to transformer the combined encodings e(vk)⁺ into the output finding w, which is either an alternative to initial finding or a confirmation of this. Like pre-processor PP, transformer TRF may be configured as a trained ML model. For example, the general setup of transformer TRF may be similar to that of combiner COM of pre-processor PP, but layers of the transformer TRF are preferably not convolutional, but are instead two or more fully connected layers, in particular if a classification result is sought, such as in classification of input feature maps from combiner COM into the output finding. If the input is to be regressed into the findings, again, fully connected layers may be used, although specially configured convolutional kernels, with spatially adapted convolutions and paddings may be used instead in some embodiments, if desired. However, a fully connected architecture is preferred herein.

More broadly, the transformer stage TRF may include an attention mechanism to learn spatial dependencies, which tend to get lost in convolutional setups. The attention mechanism may be implemented by matrix multiplication or normalization, or other. The attenuation mechanism allows re-weighting of portion(s) of sequential input so as to model language context for example. An autoencoder (AE) or variational AE (VAE) may be used. The attenuation mechanism may be implemented as a sub-model, such as fully connected layer interposed between encoder and decoder of the AE or VAE. The fully connected sub-model as attention mechanism may receive input from encoder and from output of decoder. Such an attention mechanism may provide its output as input to decoder. Variants of attention mechanisms may be implemented as dot product, query-key-values, or others. Such mechanisms recombine inputs at the encoder-side to redistribute those effects to each target output.

Preferably, combiner COM is convolutional or a hybrid, fully connected- and -convolutional, and transformer TRF is fully connected.

Transformer TRF, a component of the downstream machine learning system MLS, transforms the encoded information e(vₖ), and produces the output recommendation w. Output recommendation w may be an alternative to the initial recommendation in the report PR for example. In some cases, the output essentially w equals the initial finding, thus reassuring the user. If there is an alternative finding, the user may have a second more details look in a second review session and may then chose, as described earlier, to accept or reject the alternative finding using suitable UI arrangement, such as a GUI etc.

Transformer TRF may operate as a regression type network or as a classifier network. One such example for Transformer TRF include fully connected NNs as opposed to CNNs as may be used in the pre-processor PP architecture. BERT type networks may be used for example, such as described by A Smit et al in “CheXbert: Combining Automatic Labelers and Expert Annotations for Accurate Radiology Report Labeling Using BERT”, arXiv:2004.09167, available online at https://arxiv.org/abs/2004.09167 of 2021-10-12. Transformer TRF is preferably a language model, such as BERT or other. Its input (the encodings e(vₖ)+) are preferably transformed into text output. The transformer TRF may embed such input into a feature vector for example. Thus, transformer TRF may be described as a text-embedding language model in some embodiments.

The output of Transformer TRF is not necessarily a single finding, although this may be so in embodiments. Plural such findings may be output in embodiments. The format may be in vector form, such as some of the input (vk), and so coded that each index represents a finding, and each entry a score for that finding “i”. The entry may not necessarily be probabilities as more than one finding may be applicable. Transformer TRF may include a soft-max layer, or any another normalizer, to combine the hybrid encodings e(vk)⁺ in a manner so that each entry of the output vector can be represented as such a score. The final output as provided to user may include an indication only of the finding with the top score in the output vector. Alternatively, a list of m findings for the first m-scores (m>1) is provided. The vector index may be matched to a NL string that describes the finding in NL, such as “PNEUMONIA”, or instead a code, numerical or otherwise, may be provided. The string w may be displayed, stored, transmitted in a text message, email, sounded out, etc.

The localizer LC may be implemented as separate component or as a component included in the Transformer TRF or pre-pre-processor PP. Localizer LC is operable to identify one or more portions of the input imagery I. The identified portion includes pixels that contributed more to the output w arrived at, than did other pixels in the neighborhood or globally across the whole image plane. The contribution may be measured by thresholding or by observing a gradient behavior grad_{I}g of the whole model, comprising pre-processor PP and Transformer TRF, considered as a function w=g(I, θ*), with θ* the parameters of the model. The localizer may be implemented by heatmap technology, such as GradCAM, GradCAM++, or other class activation mapping techniques. etc. Suitably technologies envisaged herein are described by Ramprasaath R. et al. in “Grad-CAM: Visual Explanations from Deep Networks via Gradient-based Localization”, published at arxiv: 1610.02391 [cs.CV], available online at https://arxiv.org/abs/1610.02391. See also (GradCAM++) ChattopadhyayA. et al. in “Generalized Gradient-based Visual Explanations for Deep Convolutional Networks”, published at arxiv: 1710.11063 [cs.CV], available online at https://arxiv.org/abs/1710.11063v2. See also the concept of saliency maps also envisaged herein and described by Simonyan K. et al. in “Deep Inside Convolutional Networks: Visualizing Image Classification Models and Saliency Maps”, published at arXiv:1312.6034 [cs.CV], available online at https://arxiv.org/abs/1312.6034.

The identified portion may be graphically rendered by the mentioned bounding boxes, as caused by the visualizer VIZ for example, in interaction with the output w provided by recommender module MA.

The localization lc(w) of finding w may be achieved instead by training the model to output (w,p), with p a set of image co-ordinates that define the portion in the input image to which the output finding w pertains. Thus, in this embodiment, the localization information p is native. Thus, the coordinates p are provided in the training data and are considered by the objective function during training. Thus, in this embodiment, the localizer LC is a functionality implicit in the trained model PP,COM.

The alternative finding w may relate to a completely different organ or tissue type in the patient, as compared to initial/original finding. However, this may not be necessarily so. The alternative finding may still relate to the same organ as per the radiologist's initial finding, but may represent instead a different diagnosis or medical insight in relation to the same organ.

Pre-processor PP and the downstream recommender machine learning system MLS (including the Transformer TRF) may be implemented on the same processing unit, such as on a server or other computing device. However, this is not necessarily envisaged herein in all embodiments. A distributed implementation is also envisaged. For example, pre-processor PP may be implemented on one computing unit PU, whilst the downstream machine learning system MLS including the Transformer TRF is implemented on another processing unit PU′ (not shown). Preferably, the processing units PU,PU′ are communicatively coupled to one another so that the combined encodings e(vk)+ produced by the pre-processor PP may be provided for combining by the downstream machine learning system MLS. For example, processing units PU,PU′ may be geographically remote to another. Pre-processor PP may be implemented on one (or more) servers, whilst system MLS may be implemented on a user's terminal device, such as on a handheld device (laptop, smartphone etc), or on a stationary device such as a desktop, workstation, or the like. Alternatively, it is the MLS that is implemented on one or more servers, and it is the pre-processor PP that runs on such a user's terminal device.

Preferably, to achieve good throughput, the computing device PU includes one or more processors (CPU) that support parallel computing, such as those of multi-core design. In one embodiment, GPU(s) (graphical processing units) are used.

Referring now in yet more detail to the preprocessor PP in FIG. 3, in encoder portion ENC, raw input data items rk are processed in separate strands or channels (shown as a set of parallel lines to the left of FIG. 3) into respective “pure” encodings (vk), including encodings for the core data. Specifically, raw input data rk is encoded in respective pure forms in separate processing channels into respective encoded input data vk. Such pure encodings may then be combined by combiner COM into the said intermediate result e(vₖ)+, which may now be referred to as hybrid encodings e(vₖ). The intermediate encoding results e(vₖ) are a set encodings, but each is now no longer pure but includes instead cross-contributions combined-in from pure encodings from other channels/strands. This is indicated in FIG. 3 by multiple lines feedings into nodes n₃ⱼ of output OL, each line carrying contributions that originated at least in parts from some or all of the encoded input data vk. Thus, the combiner COM mixes or balances contributions from other/differentchannels vk.

The number of such combined encoding results e(vₖ)+ may vary. For dimensional-reducer type combiners COM, the number may be lower than the number of encoded items vₖ. The encoder ENC may be implemented as a mapper that maps the raw input into elements vₖ of a vector space, such as respective vectors or matrices or tensors, as the case may be.

Combiner COM then mixes or balances contributions from the pure encodings vₖ into the intermediate results which are now vector space elements e(vₖ)+ with cross-contributions from different channels. Such intermediate results may include feature maps when combiner COM is configured as a CNN. In some embodiments, some or each intermediate result e(vₖ)+ may include contributions from some or all other data items vₖ from the other channels k′!=k.

No machine learning elements are necessarily needed for the encoder portion ENC, as this could be implemented as a LUT, if length/dimension of raw data in the respective channel is known for example. The combiner COM however is preferably implemented as described above as an ML model, such as a neural network NN, preferably a CNN, fully connected network or hybrid network, including the computational nodes nᵢⱼ.

Some data type, such as patient history PH, may vary in length or dimension, which may not be known a priori. For such type of data a, preferably, ML-based, sequential data processor SDP may be provided in the strand/channel for processing the said patient history data PH. This may be implemented as a recurrent neural network, possibly of the convolutional type, or of the long short-term memory (LSTM) type. In general, RNN describe all forms of NNs that take their outputs back into their inputs. This includes networks—fully connected or convolutional—with a “looping back” facility, but also more complex architectures such as LSTM or gated recurrent units (GRU) that transmit two types of information, the so called cell states Cₜ and the output hₜ of a node in a layer. In distinction thereto, the pre-processor PP's second stage combiner COM may be configured as a feed-forward network.

The raw data (rₖ) mentioned earlier may include text data (such as in the report PR, request RQ, patient history PH, etc), numerical data such as the statistical data ST, or spatial pixel/voxel data such as the input image I. In some channels, no such encoding is needed. Such channels can be implemented as a unity operator, that passes on its input unchanged to combiner COM. For example, the input image data I may not need encoding in the first stage ENC as image data is already inherently suitably encoded as a matrix of pixel values. However, in embodiments the channel for the image channel may still include a trained ML component, such as an image classifier that classifies the image into a vector of conditions, disease etc. Thus, the input image I may be encoded into a vector. A CNN type network with a classifier output layer (a normalizer, such as soft-max layer, etc) may be used to configure such an ML component, suitably trained beforehand on some training imagery, suitably labelled for example. This classifier, and/or the sequence data processor SDP may be co-trained together with combiner model COM, and/or with the transformer model TRF. In general, ML components of the encoder stage ENC may trained beforehand or with the models for combiner COM and transformer TRF.

The encodings at encoder stage ENC, and/or the combined encodings of combiner COM may be provided in tensorial, matricidal or vector form, as the case may be. In the following examples, encodings (vₖ) for the different types of raw data (rₖ) at the first stage encoder ENC are described in vector form, with the understanding that this is exemplary and not limiting herein.

For example, Natural Language Processing (NLP) methods can be applied to retrieve the different findings from a radiologist's free hand report. A BERT type network may be used, a type of trained neural network that translates free hand reports into radiological findings, such as into a list of “Fracture, Consolidation, Enlarged Cardiomediastinum, No Finding, Pleural Other, Cardiomegaly, Pneumothorax, Atelectasis, Support Devices, Edema, Pleural Effusion, Lung Lesion, Lung Opacity”. Those findings can be encoded by encoder ENC into one-hot vectors, for instance as:

$${\vec{v}}_{RP} = [ 0 , 0 , 0 , 1 , 0 , 0 , 0 , 0 , 0 , 0 , 0 , 0 , 0 , 0 ]$$

T The position of the l's indicate the presence of the finding, in this case “No Finding”. In addition to the exploitation of the radiologist's report RP, this kind of models can be used to parse the request RQ for radiological study. By means of transfer learning, the same network, such as CheXbert or other BERT types networks can be adapted to process the request RQ for radiological study into classes, including “Airways study, Trauma, Oncology, Device verification” and others, that can as well be encoded into one-hot vectors:

$${\vec{v}}_{RQ} = [ 0 , 1 , 0 , 0 , 0 , 0 , 0 , 0 ]$$

Finally, the same or similar NLP methods can be transferred to process the patient's history PH of diseases into respective vectors. However, in this case, it would more suitable not to use one-hot vectors, since the number of diseases in each patient's history is different, and the time since remission is likely to be an informative factor. The vector for patient history PH encoding may code instead for duplets of values, one coding for the type of disease and the second encoding of the elapsed time since remission, for example:

$${\vec{v}}_{PH} = [ ( 22 , 168 ) , ( 5 , 17 ) , ( 64 , - 1 ) , … ]$$

In this example, disease code “22” has been in remission for 168 months, the disease code 5 has in remission for 17 months, whilst disease code 64 is not in remission yet as indicated by a negative number for example. Such an implementation may be based on a Look Up Table (LUT) of diseases. Such LUT may allow establishing correspondences between codes and conditions, for instance “22” may code for “cancer”, 5 for “fracture”, whilst 64 codes for “pneumonia”.

Instead of passing imagery through by identity operator as mentioned earlier, the input image I processing channel may include its own NN, such as a CNN, which can be configured to process the imagery I into a class. Again, a BERT type setup as like CheXbert may be used, but with image data as input. Output that is provided to the combiner, may be configured again as a one-hot vector, where each entry represents absence or presence of a class:

$${\vec{v}}_{I} = [ 0 , 0 , 0 , 1 , 0 , 0 , 0 , 0 , 0 , 0 , 0 , 0 , 0 , 0 ]$$

As above, the classes into which the imagery I is classified may represent medical conditions/disease etc.

Thus, it can be seen that the first stage encoder ENC may provide, using its own ML networks per channel, different vectors that code for disease/conditions and the combiner COM consolidates these “votes”, as the vector/matrices may be called, into a balanced vote with cross-contributions from different channels to obtain the hybrid encoding e(vk)+es mentioned above: Thus, second stage combiner COM may be understood as a mathematical function ƒ, where each “vote” (vₖ), encoded as vector or other indexed structure i is accounted for:

$$e ( Vk ) = f ( {\vec{v}}_{RP} , {\vec{v}}_{RQ} , {\vec{v}}_{PH} , {\vec{v}}_{I} , {\vec{v}}_{p} )$$

The localization component vₚ may also be included herein, but this is optional. The localization data may be feed into a hidden layer as shown.

The statistical data ST may be encoded as a list (vector) or matrix, including the relevant statistical descriptor (percentages for estimated mis-diagnosis probability etc, optionally including mean, variance, etc) for describing mis-diagnosis per medical condition. For example, an index of the encoding vector may relate to a respective medical condition, and an entry at that index to the statistical descriptor for the condition at that index. Although not shown in FIG. 3, there may be a channel for the statistical data ST, instead of, of in addition to any one of the context data channels.

Consistent with the notation used herein, context data channels are indicated schematically in FIG. 3 by “x”, and core data channels by “c”. The particular order of the channels (from top to bottom) in FIG. 3 is immaterial.

Reference is now made to FIG. 4 which shows a training system TS that may be used to train the machine learning architecture described in FIG. 3.

The pre-processor PP (encoder and/or combiner) and the downstream transformer TRF may be trained together as a whole (and this is may be preferred in some circumstances), or may be trained separately. In the latter, transfer training setups may be used. Training together or separately may depend on the exact structure of the loss function, and either option is specifically envisaged herein in embodiments.

It should also be understood that the input data in training or deployment or testing may be provided separately in different channels as matrix or vector data as described. However, in alternative embodiments, these may be consolidated into multi-dimensional “cubes” (tensors), for example, and then processed together.

As mentioned earlier, training data may be sourced from medical data bases TD that may include patient records from previous patients, preferably from a large number of patients in a suitably varied cohort. v˜ indicates training input data, corresponding to (vk) of FIG. 3, whilst w˜ indicates the associated findings (alternative or confirmatory) from historical cases that can serve as training labels in a supervised machine learning setup, preferably envisaged herein, although unsupervised training setups are not excluded herein.

Such alternative findings may be found as mentioned in medical audit databases where mis-diagnosed cases are recorded that have been investigated in the past. These records most likely include the correct (alternative) finding which can be used as a label w˜ associated with the training input data v˜. The training input data v˜ on the other hand incudes the earlier mentioned core elements, such as the initial report that was filed, including the initial false or incorrect finding, and the image data on which it was based. As mentioned earlier, suitable one or more of the mentioned contextual data c may be used herein. Thus, the training data includes instances of respective historical initial finding (which may be wrong) and the later, correct finding so that the training system TS is capable of learning the above mentioned latent relationship. Preferably, the training data includes a number of correct cases where the findings in input v˜ and label w˜ are identical, for better robustness and learning performance. Thus, the training system TS is also exposed to samples of “correct” diagnostic training material for better separation from material that represent misdiagnoses.

It will be appreciated, that inclusion, as contextual data, of the statistical data ST for mis-diagnoses per finding types as mentioned earlier as a separate input channel into the machine learning model helps achieve particular robustness, accuracy and quick learning. However, the statistical data ST is in general no longer need in deployment or testing. The same applies to the other types of contextual data c. Application of the core input in deployment suffices, although user may still choose to “reopen” additional one or more input channels so as to add in some embodiments context data in deployment, if required to boost performance in some cases.

Two processing phases may thus be defined in relation to the machine learning models: a training phase and a later deployment (or inference) phase.

In training phase, prior to deployment phase, the model is trained by adapting its parameters based on the training data. Once trained, the model may be used in deployment phase to compute the alternative findings, if any. The training may be a one-off operation, or may be repeated once new training data become available.

In the training phase, an architecture of a machine learning model M=PP,TRF such as shown the NN network in FIG. 3 is pre-populated with initial set of weights. The weights θ of the model NN represent a parameterization M^{θ}, and it is the object of the training system TS to optimize and hence adapt the parameters θ based on the training data (v˜ₖ, w˜ₖ) pairs. In other words, the learning can be formulized mathematically as an optimization scheme where a cost function F is minimized although the dual formulation of maximizing a utility function may be used instead.

Training is the process of adapting parameters of the model based on the training data. An explicit model is not necessarily required as in some examples it is the training data itself that constitutes the mode, such as in clustering techniques or k-nearest neighbors. etc. In explicit modelling, such as in NN-based approaches and many others, the model may include as system of model functions/computational nodes, with their input and/or output it least partially interconnected. The model functions or nodes are associated with parameters θ which are adapted in training. The model functions may include the convolution operators and/or weights of the non-linear units such as a RELU, mentioned above at FIG. 3 in connection with NN-type models. In the NN-case, the parameters θ may include the weights of convolution kernels of operators CV and/or of the non-linear units. The parameterized model may be formally written as M^{θ}=PP, TRF. The parameter adaptation may be implemented by a numerical optimization procedure. The optimization may be iterative. An objective function F may be used to guide or control the optimization procedure. The parameters are adapted or updated so that the objective function is improved. The input training data v˜ⁱ is applied to the model. The model responds to produce training data output M(v˜ⁱ)=\tilde{w}ⁱ. The objective function is mapping from parameters space into a set of numbers. The objective function ƒ measures a combined deviation between the training data outputs \tilde{y}ⁱ and the respective targets w˜ⁱ. Parameters are iteratively adjusted to that the combined deviation decreases until a user or designer pre-set stopping condition is satisfied. The objective function may use a distance measure ∥·∥ to quantify the deviation.

In some embodiments, but not all, the combined deviation may be implemented as a sum over some or all residues based on the training data instances/pairs (v-˜ⁱw˜ⁱ)ⁱ, and the optimization problem in terms of the objective function may be formulated as:

$$arg {min}_{θ} F = {\sum }_{k} ‖ {M}^{θ} ( {x}_{k} ) , {y}_{k} ‖ \qquad ( 1 )$$

In the setup (1), the optimization is formulated as a minimization of a cost function F, but is not limiting herein, as the dual formulation of maximizing a utility function may be used instead.

Summation is over training data instances i.

The cost function F may be pixel/voxel-based, such as the L1—or the smoothed L1-norm, L2-norm, Hubert, or the Soft Margin cost function. For example in least squares or similar approaches, the (squared) Euclidean-distance-type cost function in (1) may be used for the above mentioned regression task for regression into output finding(s). When configuring the model as a classifier that classifies into findings, the summation in (1) is formulated instead as one of cross-entropy or Negative Log Likelihood (NLL) divergence or similar.

The exact functional composition of the updater UP depends on the optimization procedure implemented. For example, an optimization scheme such as backward/forward propagation or other gradient based methods may then be used to adapt the parameters θ of the model M so as to decrease the combined residue for all or a subset (v˜ₖ, w˜ₖ) of training pairs from the full training data set. Such subsets are sometime referred to as batches, and the optimization may proceed batchwise until all of the training data set is exhausted, or until a predefined number of training data instances have been processed.

Training may be a one-off operation, or may be repeated once new training data become available.

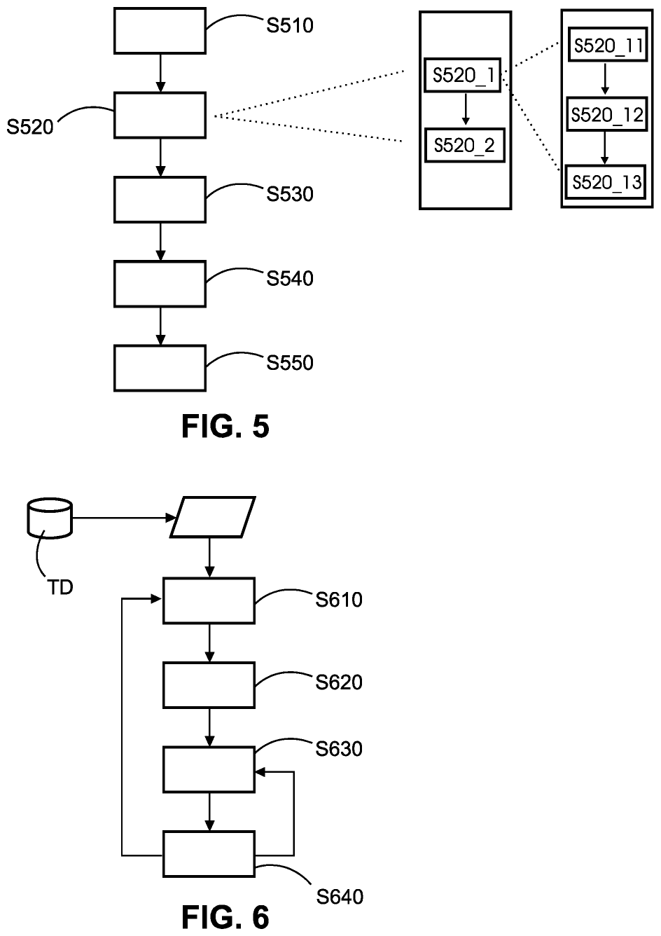

Reference is now made to FIG. 5 which shows a flow diagram of a computer-implemented method of computing confirmatory or alternative findings w, based on in particular core input data c including a user generated initial finding (such as in medical report PR), and image data I on which the user generated finding was based. Optionally, contextual data x may be used alongside core data c. It will be understood, however, that the method described herein is not necessarily tied to the architectures described above, and the below may be understood as a teaching in its own right. The method is preferably based on ML models. It is assumed that such models have been trained on training data.

At step S510, during testing or deployment, input data is received including the said initial finding and its associated imagery I. Optionally, contextual data of any of the above types may be used in addition.

More particular still, at step S510 input data is received such as a report including the initial finding generated by the radiologist and the medical image I to which the report pertains and on which it is based. Alternatively, the original finding is extracted from the report first, and the so extracted finding is received. Thus it is not necessary herein to process all the report although this can still be done as the report itself may include useful contextual data.

At step S520 the input data is co-processed by a machine learning model to produce an output finding. The output finding may be an alternative to the one included in the initial input data, or may be a confirmation thereof.

The alternative finding, if any, is then output at step S530. Alternatively, the output finding confirms (is equal to) to the initial finding, and this too may be output.

In step S540, the output finding is displayed, sounded out, or otherwise brought to the attention of user by controlling a suitable transducer based on computed output finding w.

The machine learning model(s) as used at step S520 may implement a two-step operation, including first a pre-processing S520_1 and then a transforming S520_2 operation. The pre-processing in turn may include an encoding operation S520_11 and then a combiner operation S520_12. In particular, the pre-processing may include using a ML model to encode (raw) input data by an encoder portion of model. The encoded data is then combined into combined encoded data by a combiner portion of the model. The combined encoded is then provided S520_13 for further ML processing. Specifically, it is then in a second step S520_2 that the combined encoded data is transformed by a transformer stage of the machine learning model.

The transform operation transforms the combined encoded output into the output finding. The transforming may include a regression or classification into textual or coded data, as applicable, which is indicative of the output finding. The output finding may be an alternative finding or a confirmation of the initial finding.

The pre-processing and the transforming operation may be each configured as a separate ML model. The two models may be thought of parts of a super-model comprising the two models. Some or each model may include sub-model(s). The encoder of pre-processing model may include in parts ML models (such as sequential data processing or image classifier) and the combiner may include a separate ML model. The transformer model may in itself be implemented as a sub-ML-model. Thus, the super-ML model may include plural ML sub-models, nested and/or in sequence.

The combined encoded data produced by step S520_12 includes cross-contribution from both, the input image and the initial finding, and, optionally, from context data. For example, the combined encoded data may be represented as feature maps in a CNN or other NN model. The encoder encodes the data into matricial or vector form. The encoder may used at least in parts for some or each channel as separate one-hot encoding scheme as described above. The transform operation or combiner may include weighted linear combinations of logits from a previously layer, with a non-linearity applied to produce a contribution which may then be passed to nodes of the next layer.

A recurrent network may be used in the encoding operation to process part of the input data including in particular patient record history data as this has variable size or dimension. Other data type in different channels whose expected dimension/size is variable, such as the length of the historical data etc, may also be processed thus.

The encoding operation may include classifying the input image (to which the initial finding pertains) into a classification vector. An index of this vector may represent a medical condition, etc, and each entry a corresponding score of the respective condition. This vector may be “one-hotted” (binarized) by setting the index with maximum score as “1”, and nullifying all remaining entries.

As an optional step S550, the computed alternative finding is localized in the input image of the initial input data. This may include using heat-map techniques or similar where a contribution to the final result (the alternative finding) of pixels or pixel regions in the input image is measured and the localization indicates those image pixel region or regions that contributed more than a given threshold as compared to remaining pixels. Gradient based techniques applied to the ML model, treated as differentiable function, may be used as described above.

The output, that is the confirmatory or alternative finding, may be in textual form or may be in a medical code or other.

Referring now to a block diagram in FIG. 6, this shows a flow chart of a method of training the above described machine learning models PP, COM. Preferably both are trained together. The training data v˜, w˜ is a described above in FIG. 4.

At step S610 training data is received in the form of pairs (v˜ₖ,w˜ₖ). Each pair includes the training input xₖ and the associated target w˜ₖ. v˜ₖ, as defined in FIG. 5 above.

At step S620, the training input v˜ₖ is applied to an initialized machine learning model M to produce a training output.

A deviation, or residue, of the training output M(v˜ₖ) from the associated target w˜ₖ is quantified at S630 by a cost function F. One or more parameters of the model are adapted at step S640 in one or more iterations in an inner loop to improve the cost function. For instance, the model parameters are adapted to decrease residues as measured by the cost function. The parameters include in particular weights of the convolutional operators, in case a convolutional model M is used.

The training method then returns in an outer loop to step S610 where the next pair of training data is fed in. In step S620, the parameters of the model are adapted so that the aggregated residues of all pairs considered are decreased, in particular minimized. The cost function quantifies the aggregated residues. Forward-backward propagation or similar gradient-based techniques may be used in the inner loop.

Examples for gradient-based optimizations may include gradient descent, stochastic gradient. conjugate gradients, Maximum likelihood methods, EM-maximization, Gauss-Newton, and others. Approaches other than gradient-based ones are also envisaged, such as Nelder-Mead, Bayesian optimization, simulated annealing, genetic algorithms, Monte-Carlo methods, and others still.

More generally, the parameters of the model NN are adjusted to improve objective function F which is either a cost function or a utility function. In embodiments, the cost function is configured to the measure the aggregated residues. In embodiments the aggregation of residues is implemented by summation over all or some residues for all pairs considered. In particular, in preferred embodiments the outer summation proceeds in batches (subset of training instances), whose summed residues are considered all at once when adjusting the parameters in the inner loop. the outer loop then proceeds to the next batch and so for until the requisite number of training data instances have been processed. Instead of processing pair by pair, the outer loop access plural pair of training data items at once and looping is batchwise. The summation over index “k” in eq (1) above may thus extend batchwise over the whole respective batch.

Although in the above main reference has been made to NNs, the principles disclosed herein are not confined to NNs. For example, instead of using the NN for pre-processor PP, another approach such as Hidden Markov Models (HMM), or Sequential Dynamical Systems (SDS), among which especially Cellular Automata (CA) may be used.

Similarly, For example, instead of using the NN for combiner COM, another ML approach such as Support Vector Machines (SVM), or boosted decision trees may be used.

The components of the recommender MA may be implemented as one or more software modules, run on one or more general-purpose processing units PU such as a workstation associated with the imager IA, or on a server computer associated with a group of imagers.

Alternatively, some or all components of the recommender MA may be arranged in hardware such as a suitably programmed microcontroller or microprocessor, such an FPGA (field-programmable-gate-array) or as a hardwired IC chip, an application specific integrated circuitry (ASIC), integrated into the imaging system IA. In a further embodiment still, the recommender MA may be implemented in both, partly in software and partly in hardware.

The different components of the recommender MA may be implemented on a single data processing unit PU. Alternatively, some or more components are implemented on different processing units PU, possibly remotely arranged in a distributed architecture and connectable in a suitable communication network such as in a cloud setting or client-server setup, etc.

One or more features described herein can be configured or implemented as or with circuitry encoded within a computer-readable medium, and/or combinations thereof. Circuitry may include discrete and/or integrated circuitry, a system-on-a-chip (SOC), and combinations thereof, a machine, a computer system, a processor and memory, a computer program.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above-described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium (in particular, but not necessarily, a non-transitory medium), such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope. Such reference signs may be comprised of numbers, of letters or of any alphanumeric combination.

1. A machine learning arrangement for processing medical data, comprising a pre-processor component and a machine learning system,
wherein the pre-processor component comprises:
at least one input interface for receiving a human user generated initial finding for a patient and a medical image to which the said finding pertains;
an encoder for encoding the finding and the medical image into encoded data, including encoded image data and encoded finding data;
a combiner for combining the encoded finding and the encoded image data into combined encoded data;
an output interface for providing the combined encoded data to the machine learning system, and
wherein the machine learning system includes a machine learning model configured to transform the combined encoded output into output data that is indicative of at least one second finding, the at least one second finding being an alternative to the initial finding, wherein the at least one alternative finding indicating a condition or disease, and wherein the arrangement causes the at least one alternative finding to be brought to the attention of the user.

2. The arrangement of claim 1, wherein the input interface is configured to receive contextual data, providing context information in relation to the report and/or the image, the encoder is configured to encode at least a part of the contextual data into the encoded data, and the combiner is configure to combine the encoded contextual data with the image and the encoded report to obtain the combined data.

3. The arrangement of claim 1, wherein the contextual data includes at least one of: i) the patient history, ii) an imaging request for the image, iii) statistical data in relation to misdiagnosis.

4. The arrangement of claim 1, wherein the combiner and/or the encoder is implemented as a respective machine learning model.

5. The arrangement of claim 4, wherein the machine learning model for the encoder includes a processing channel configured for recurrent processing.

6. The arrangement of claim 5, wherein the processing channel is configured to process at least the encoded patient history.

7. (canceled)

8. (canceled)

9. The arrangement of claim 1, wherein the output includes a natural textual string or a medical finding code.

10. The arrangement of claim 9, further comprising a localizer configured to map the output data to an image location in the image.

11. (canceled)

12. A method for pre-processing medical data for machine learning, comprising:
receiving a human user generated initial finding for a patient and a medical image to which the said finding pertains;
encoding the finding and the medical image into encoded data, including encoded image data and encoded finding data;
combining the encoded finding and the encoded image data into combined encoded data;
providing the combined encoded data to the machine learning system;
transforming, by the machine learning system, the combined encoded output into output data that is indicative of at least one second finding, the at least one second finding being an alternative to the initial finding, wherein the at least one alternative finding indicates a condition or disease; and
bringing the at least one alternative finding to the attention of the user.

13. (canceled)

14. (canceled)

15. A non-transitory computer readable medium having stored thereon executable instructions that, when executed, cause the method of claim 12 to be performed.
